SEQUENCE LISTING (63 sequences)

SEQ ID NO: 1 - protein, 33 aa, Unknown
  Description of Unknown Orexin-A polypeptide
EPLPDCCRQKTCSCRLYELLHGAGNHAAGILTL

SEQ ID NO: 2 - DNA, 25 bp, Artificial Sequence
  Description of Artificial Sequence Synthetic primer
tggctgtctgtatagttgctatgat

SEQ ID NO: 3 - DNA, 21 bp, Artificial Sequence
  Description of Artificial Sequence Synthetic primer
cgagaccttccagatcacagt

SEQ ID NO: 4 - DNA, 22 bp, Artificial Sequence
  Description of Artificial Sequence Synthetic primer
tgccacaacccaaccagtctca

SEQ ID NO: 5 - DNA, 20 bp, Artificial Sequence
  Description of Artificial Sequence Synthetic primer
acaggcaggctaagaaccag

SEQ ID NO: 6 - DNA, 20 bp, Artificial Sequence
  Description of Artificial Sequence Synthetic primer
ttccctcctgcaagctctac

SEQ ID NO: 7 - DNA, 19 bp, Artificial Sequence
  Description of Artificial Sequence Synthetic primer
ctgaggacgcaaaatgagg

SEQ ID NO: 8 - DNA, 19 bp, Artificial Sequence
  Description of Artificial Sequence Synthetic primer
ccagccaaaactcccactt

SEQ ID NO: 9 - DNA, 18 bp, Artificial Sequence
  Description of Artificial Sequence Synthetic primer
tcctctgaaaccggatgg

SEQ ID NO: 10 - DNA, 20 bp, Artificial Sequence
  Description of Artificial Sequence Synthetic primer
caaaggatgattcggctcag

SEQ ID NO: 11 - DNA, 19 bp, Artificial Sequence
  Description of Artificial Sequence Synthetic primer
aaacaacgcaacgtggaga

SEQ ID NO: 12 - DNA, 22 bp, Artificial Sequence
  Description of Artificial Sequence Synthetic primer
gaaagacaacggacaaatcacc

SEQ ID NO: 13 - DNA, 20 bp, Artificial Sequence
  Description of Artificial Sequence Synthetic primer
ggcctctacgactcagtcca

SEQ ID NO: 14 - DNA, 19 bp, Artificial Sequence
  Description of Artificial Sequence Synthetic primer
aaacaacgcaacgtggaga

SEQ ID NO: 15 - DNA, 18 bp, Artificial Sequence
  Description of Artificial Sequence Synthetic primer
tgatgcaatccggatcaa

SEQ ID NO: 16 - DNA, 20 bp, Artificial Sequence
  Description of Artificial Sequence Synthetic primer
cggaaatcatatccaaccag

SEQ ID NO: 17 - DNA, 18 bp, Artificial Sequence
  Description of Artificial Sequence Synthetic primer
aaggtccaggttccagca

SEQ ID NO: 18 - DNA, 21 bp, Artificial Sequence
  Description of Artificial Sequence Synthetic primer
agcacaggaatgtcgaagaaa

SEQ ID NO: 19 - DNA, 20 bp, Artificial Sequence
  Description of Artificial Sequence Synthetic primer
cggctaggactggacaactc

SEQ ID NO: 20 - DNA, 18 bp, Artificial Sequence
  Description of Artificial Sequence Synthetic primer
cgcctgggattcctctgt

SEQ ID NO: 21 - DNA, 19 bp, Artificial Sequence
  Description of Artificial Sequence Synthetic primer
aacaaccgtatgcccatga

SEQ ID NO: 22 - DNA, 20 bp, Artificial Sequence
  Description of Artificial Sequence Synthetic primer
cgggaaattctgcgaaatag

SEQ ID NO: 23 - DNA, 19 bp, Artificial Sequence
  Description of Artificial Sequence Synthetic primer
ggcgctcctgttcttccta

SEQ ID NO: 24 - DNA, 18 bp, Artificial Sequence
  Description of Artificial Sequence Synthetic primer
ctggacacggccaaagtc

SEQ ID NO: 25 - DNA, 24 bp, Artificial Sequence
  Description of Artificial Sequence Synthetic primer
gacaggaaagacaacagacaaatc

SEQ ID NO: 26 - DNA, 20 bp, Artificial Sequence
  Description of Artificial Sequence Synthetic primer
tccatgctgttatgggtgaa

SEQ ID NO: 27 - DNA, 20 bp, Artificial Sequence
  Description of Artificial Sequence Synthetic primer
tacactgtgcaggcaggcta

SEQ ID NO: 28 - DNA, 18 bp, Artificial Sequence
  Description of Artificial Sequence Synthetic primer
ggggacctgaaccacctc

SEQ ID NO: 29 - DNA, 20 bp, Artificial Sequence
  Description of Artificial Sequence Synthetic primer
gacaatgacgctgtgtctgg

SEQ ID NO: 30 - DNA, 18 bp, Artificial Sequence
  Description of Artificial Sequence Synthetic primer
cttgtgcagcgggaaact

SEQ ID NO: 31 - DNA, 18 bp, Artificial Sequence
  Description of Artificial Sequence Synthetic primer
ccatctggtggcctgaag

SEQ ID NO: 32 - DNA, 20 bp, Artificial Sequence
  Description of Artificial Sequence Synthetic primer
tacgcctgcttcaccttctc

SEQ ID NO: 33 - DNA, 27 bp, Artificial Sequence
  Description of Artificial Sequence Synthetic primer
tgcttgagatactcttacaataatgct

SEQ ID NO: 34 - DNA, 20 bp, Artificial Sequence
  Description of Artificial Sequence Synthetic primer
cagcaagaagaggtccgact

SEQ ID NO: 35 - DNA, 22 bp, Artificial Sequence
  Description of Artificial Sequence Synthetic primer
agcagtctccagcagcccaaag

SEQ ID NO: 36 - DNA, 21 bp, Artificial Sequence
  Description of Artificial Sequence Synthetic primer
cgtggagaggagtgtcttcag

SEQ ID NO: 37 - DNA, 20 bp, Artificial Sequence
  Description of Artificial Sequence Synthetic primer
cagagcgctggtcatgtagt

SEQ ID NO: 38 - DNA, 23 bp, Artificial Sequence
  Description of Artificial Sequence Synthetic primer
tggcttctggtagatgagctaaa

SEQ ID NO: 39 - DNA, 19 bp, Artificial Sequence
  Description of Artificial Sequence Synthetic primer
gaaccatgaagccaacgac

SEQ ID NO: 40 - DNA, 20 bp, Artificial Sequence
  Description of Artificial Sequence Synthetic primer
tcccacacagagggatatgg

SEQ ID NO: 41 - DNA, 24 bp, Artificial Sequence
  Description of Artificial Sequence Synthetic primer
aagctgaatatatgcctgcttttc

SEQ ID NO: 42 - DNA, 19 bp, Artificial Sequence
  Description of Artificial Sequence Synthetic primer
gcggtcattgtcactggtc

SEQ ID NO: 43 - DNA, 22 bp, Artificial Sequence
  Description of Artificial Sequence Synthetic primer
gggggtgatatgtttgaacttg

SEQ ID NO: 44 - DNA, 20 bp, Artificial Sequence
  Description of Artificial Sequence Synthetic primer
taagccggctgagatcttgt

SEQ ID NO: 45 - DNA, 19 bp, Artificial Sequence
  Description of Artificial Sequence Synthetic primer
gcggtcattgtcactggtc

SEQ ID NO: 46 - DNA, 19 bp, Artificial Sequence
  Description of Artificial Sequence Synthetic primer
cacgtgtgttgcgtcagtc

SEQ ID NO: 47 - DNA, 21 bp, Artificial Sequence
  Description of Artificial Sequence Synthetic primer
tgaggaccgctagcaagtttg

SEQ ID NO: 48 - DNA, 24 bp, Artificial Sequence
  Description of Artificial Sequence Synthetic primer
ggtatcattattagcaagccctgt

SEQ ID NO: 49 - DNA, 19 bp, Artificial Sequence
  Description of Artificial Sequence Synthetic primer
ttgagctgggtcatgcagt

SEQ ID NO: 50 - DNA, 19 bp, Artificial Sequence
  Description of Artificial Sequence Synthetic primer
ctgacagctcgcattgctc

SEQ ID NO: 51 - DNA, 21 bp, Artificial Sequence
  Description of Artificial Sequence Synthetic primer
ctggttttccgatcacactct

SEQ ID NO: 52 - DNA, 20 bp, Artificial Sequence
  Description of Artificial Sequence Synthetic primer
acatagatgaggctcaggat

SEQ ID NO: 53 - DNA, 20 bp, Artificial Sequence
  Description of Artificial Sequence Synthetic primer
tgtgcaggagcattcgtact

SEQ ID NO: 54 - DNA, 18 bp, Artificial Sequence
  Description of Artificial Sequence Synthetic primer
gtcgttgggtgctgacct

SEQ ID NO: 55 - DNA, 26 bp, Artificial Sequence
  Description of Artificial Sequence Synthetic primer
gacacctttatacctaataacactgg

SEQ ID NO: 56 - DNA, 21 bp, Artificial Sequence
  Description of Artificial Sequence Synthetic primer
ggggtgatgtgtttgaacttg

SEQ ID NO: 57 - DNA, 21 bp, Artificial Sequence
  Description of Artificial Sequence Synthetic primer
tgtgtcaaccatggtcatttc

SEQ ID NO: 58 - DNA, 21 bp, Artificial Sequence
  Description of Artificial Sequence Synthetic primer
gtgtggagaggagtgtcttcg

SEQ ID NO: 59 - DNA, 18 bp, Artificial Sequence
  Description of Artificial Sequence Synthetic primer
aacatctcccgcacgttg

SEQ ID NO: 60 - DNA, 21 bp, Artificial Sequence
  Description of Artificial Sequence Synthetic primer
cccaggcttcttggtcttaat

SEQ ID NO: 61 - DNA, 25 bp, Artificial Sequence
  Description of Artificial Sequence Synthetic primer
tcctatgttaccatttattgtgtgg

SEQ ID NO: 62 - DNA, 19 bp, Artificial Sequence
  Description of Artificial Sequence Synthetic primer
gtagtgcctgggtccatga

SEQ ID NO: 63 - DNA, 20 bp, Artificial Sequence
  Description of Artificial Sequence Synthetic primer
taaactgctcccggaatttg

(19) United States
(12) Patent Application Publication
(10) Pub. No.: US 2012/0190615 A1
(43) Pub. Date: Jul. 26, 2012

(54) METHODS AND COMPOSITIONS FOR INDUCING WEIGHT LOSS

(75) Inventors: Devanjan Sikder, Orlando, FL (US); Dyan Sellayah, Orlando, FL (US)

(73) Assignee: Sanford-Burnham Medical Research Institute

(21) Appl. No.: 13/353,174

(22) Filed: Jan. 18, 2012

Related U.S. Application Data
(60) Provisional application No. 61/434,817, filed on Jan. 20, 2011.

(51) Int. Cl. A61K 38/22; A61P 3/10; A61P 3/04

(52) U.S. Cl. 514/5.2; 514/6.9

(57) ABSTRACT

The present invention provides compositions and methods for inducing weight loss, preventing weight gain, and/or treating obesity-related conditions such as diabetes by inducing the production of brown adipose tissue in subjects by administering orexin or biologically active fragments thereof.

### CROSS-REFERENCE TO RELATED APPLICATIONS

This application claims benefit of U.S. Provisional Application 61/434,817, filed Jan. 20, 2011, which is incorporated by reference in its entirety.

### FIELD OF THE INVENTION

The present invention relates to methods and compositions for inducing weight loss and/or preventing obesity.

### BACKGROUND OF THE INVENTION

The following discussion of the background of the invention is merely provided to aid the reader in understanding the invention and is not admitted to describe or constitute prior art to the present invention.

Obesity is a medical condition in which excess body fat has accumulated to the extent that it may have an adverse effect on health, leading to reduced life expectancy and/or increased health problems. As defined by the World Health Organization, a body mass index (BMI) (measurement which compares weight and height) of between 25 kg/m² and 30 kg/m² qualifies as overweight, and a BMI of greater than 30 kg/m² qualifies as obese. Obesity increases the likelihood of development of other diseases including heart disease, type 2 diabetes, certain types of cancer, and osteoarthritis. 1.1 billion adults and 10% of children are estimated to suffer from obesity worldwide. For a complete discussion, see, e.g., Haslam D W, James W P (2005), Obesity, Lancet 366 (9492): 1197-209. Obesity may further lead to glucose intolerance as well as insulin resistance in adipose tissue, liver, and muscle, which may contribute to a host of related conditions.

Traditionally, appetite suppressing pathways have been the focal point of anti-obesity drug development, since obesity is thought to be due to excess energy intake over energy expenditure. Limiting the caloric intake, however, induces compensatory adaptations that resist weight loss. Because nutrient-sensing neurons cross talk with cognitive and behavioral components, appetite suppressants tend to produce unacceptable psychiatric side effects. However, because of the complexity of the regulation of adipogenesis, few other pathways have been explored.

Adipogenesis is a highly regulated process, involving many positive and negative regulators including hormone and nutritional signals, which involves the differentiation of preadipocytcs into adipocytes. Undifferentiated cells abundantly express Necdin, preadipocyte factor-1, and Wnt10a, among other regulators, all of which inhibit early adipogenic events. Additional known inhibitors of the preadipocyte-adipocyte transition for white fat cells include the Wnt family of proteins, preadipocyte factor-1 (or Pref-1), Gata 3, and the retinoblastoma family of proteins. See, e.g., Khan et al., U.S. Published Application No. 2006/0223104. Less is known, however, about brown adipocyte differentiation.

Three features distinguish brown adipose tissue (BAT), which mediates energy expenditure, from white adipose tissue (WAT), which is the primary fat storage site: the appearance of multilocular oil droplets, mitochondrial enrichment, and Ucp-1 expression. The balance between activities of these two types of fat cells breaks down as obesity develops. Manipulation of brown fat activity is therefore attractive from a therapeutic standpoint, given the discovery of BAT in adult humans.

Some studies have reported that obese subjects may harbor immature brown preadipocytes that lack functional β₃-adenoreceptors, and therefore do not respond to β₃ stimulation, rendering that pathway less desirable for weight loss drug development. Therefore, there is a need for alternate anti-obesity strategies that do not rely on reducing food intake, and, further, may reduce adiposity without inducing anorexia or physical activity.

### SUMMARY OF THE INVENTION

It has now been shown that administration of orexin, a neuropeptide whose depletion leads to paradoxical manifestation of obesity in the face of hypophagia, permits weight loss under conditions of caloric excess and without elevated physical activity by increasing brown fat differentiation and activity.

Therefore, one aspect of the present invention is directed to a method for inducing weight loss in a subject by administering to the subject a therapeutically effective amount of a pharmaceutical formulation containing orexin, or a biologically active fragment thereof, and a pharmaceutically acceptable carrier. Another aspect of the present invention is directed to a method for treating diabetes by administering, to a subject diagnosed as having diabetes, a therapeutically effective amount of a pharmaceutical formulation containing orexin, or a biologically active fragment thereof, and a pharmaceutically-acceptable carrier. In another aspect, the invention provides a method for preventing diabetes in a pre-diabetic subject by administering to that subject a pharmaceutical formulation containing orexin, or a biologically active fragment thereof, and a pharmaceutically acceptable carrier. In another aspect, the present invention provides a method for inducing brown preadipocyte differentiation in a subject, by administering to the subject a biologically effective amount of a pharmaceutical formulation comprising orexin or a biologically active fragment thereof and a pharmaceutically-acceptable carrier. In still another aspect, the present invention provides a method of preventing weight gain by administration of a therapeutically effective amount of a pharmaceutical formulation comprising orexin or a biologically active fragment thereof and a pharmaceutically acceptable carrier.

In some embodiments of the foregoing methods, orexin administration at a dose of about 1 mg/kg to about 100 mg/kg. Pharmaceutical formulations used in the invention may be administered orally, parenterally, by intravenous injection, intramuscular injection, subcutaneous injection, or intrathecal injection. The administration may, in some embodiments, take place between 1 and 4 times per day and may continue for at least about one week, one month, one year, or for the lifetime of the subject.

In some embodiments, the expression of Necdin, Pref-1, or Wnt 10a is reduced in the brown preadipocyte cells of the subject. Such a reduction may be by at least 10%. In further embodiments, the expression of C/ebp, Prdm16, Ppar-gamma, Foxe2, or Zfp423 is increased in the brown preadipocyte cells of the subject. Such an increase may be by at least 10%.

By “treating” is meant the medical management of a subject with the intent that a cure, amelioration, or prevention of obesity or a related or accompanying disorder will result. This term includes active treatment, that is, treatment directed specifically toward improvement of obesity, and also includes causal treatment, that is, treatment directed toward removal of the cause of the disease, pathological condition, or disorder. In addition, this term includes palliative treatment, that is, treatment designed for the relief of symptoms rather than the curing of the disease: preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “a therapeutically effective amount” is meant the amount of a compound, alone or in combination with another therapeutic regimen, required to treat, prevent, or reduce obesity or an accompanying disease such as diabetes in a clinically relevant manner. A sufficient amount of an active compound used to practice the present invention for therapeutic treatment of conditions affecting weight gain varies depending upon the manner of administration, the age, body weight, and general health of the subject.

As used herein, “transcriptional regulators” and “adipogenic regulators” are used interchangeably to refer to genes involved in controlling expression of one or more genes indicated in adipogenesis, differentiation of preadipocytes, or related processes. Such genes may include, but are not limited to, C/epb, C/epb-α, Prdm16, Pgc-1, PPAR-γ, Foxe2, and/or Zfp423.

As used herein, “subject” refers to a mammal (e.g., human, dog, cat, and horse) that is suffering from obesity or a related or accompanying disorder or is identified as having an increased likelihood of developing obesity or a related or accompanying disorder.

As used herein, “biologically active fragments” refers to polypeptides having greater than 95% amino acid sequence identity with all or part of the amino acid sequence encoding Orexin-A, and wherein the all or part of the amino acid sequence encoding Orexin-A retains some or all of the biological function of the complete Orexin-A neuropeptide.

### BRIEF DESCRIPTION OF THE FIGURES

FIG. 1 is a series of photomicrographs of hematoxylin and eosin stained intrascapular BAT (iBAT) from wildtype mice and transgenic mice lacking orexin (OX KO), OXR1, or OXR2 at (a) 6-8 weeks of age and (b) in newborn mice.

FIG. 2 is a bar graph illustrating the effects of OX, OXR1 and OXR2 deficiency on triglyceride stores as assessed by iBAT glycerol release.

FIG. 3 is a bar graph showing the relative mRNA expression of the indicated genes in iBAT of wildtype, OX KO, OXR1 KO and OXR2KO mice.

FIG. 4 is a graph showing the Ct values from a qPCR analysis of mesenchymal stem cells in which the orexin receptor (OXR1) is expressed.

FIG. 5 illustrates immunoblotting of proteins functioning in adipogenesis with antibodies against C/ebp-α, Ppar-γ1, Prdm16, Pgc1-a, and Ucp1 following differentiation of mesenchymal stem cells.

FIG. 6 is a series of bar graphs showing the PCR analysis of adipogenic inhibitors in C3H10T1/2 cells treated with OX. Results are expressed as arbitrary units after normalization to 18S RNA.

FIG. 7 is a series of photomicrographs of cultured primary brown preadipocytes stained with Oil Red O showing the lipid accumulation following differentiation induced by either OX or BMP-7.

FIG. 8 is a series of photomicrographs of cultured mouse embryonic fibroblasts (MEFs) stained with Oil Red O showing elevated lipidogenesis following OX or BMP-7 treatment.

FIG. 9 is a series of graphs quantifying the relative expression of RNA of genes regulating adipogenesis in a culture of cells treated with OX or BMP-7.

FIG. 10 is a series of graphs quantifying the relative express on of RNA of early adipogenic inhibitors in a culture of cells treated with OX or BMP-7.

FIG. 11 is a bar graph showing the relative expression of RNA coding for adipogenesis markers in a culture of cells treated with OX or BMP-7.

FIG. 12 is a series of photomicrographs illustrating cellular differentiation, lipidogenesis, and mitochondrial biogenesis following OX or BMP-7 treatment.

FIG. 13 is a bar graph showing the oxygen consumption rates of vehicle-, OX-, and BMP-7-treated cells in the absence (basal) presence of oligomycin or FCCP or cAMP.

FIG. 14 is a graph showing the Ct value determined using, qPCR for OXR1 expressed in HIB1 preadipocyte cell line.

FIG. 15 is a series of photomicrographs of HIB1 cells stained with Oil Red O following OX or BMP-7 treatment which demonstrates lipid accumulation accompanying cellular differentiation.

FIG. 16 is a bar graph quantifying the relative expression of RNA coding for regulators of adipogenesis in HIB1 cells cultured in the presence of OX or BMP-7.

FIG. 17 is a photomicrograph of culture dishes containing cultured mesenchymal stem cells following transfection with lentivirus stably expressing orexin (Len-OX) compared to vector controls in the absence or presence of exogenous OX.

FIG. 18 is a magnified photomicrograph of Oil Red O stained HIB1 brown preadipocytes with OXR1 knocked out by infection with lentivirus containing shRNA targeting OXR1 or control vector.

FIG. 19 is a series of photomicrographs showing mitochondrial and nuclear staining in OXR1 lentivirus KO HIB1 brown preadipocytes under a variety of culture conditions.

FIG. 20 is a series of photomicrographs demonstrates lipid accumulation in primary brown preadipocytes crom wild-type and OXR1 KO mice via Oil Red O staining.

FIG. 21 is a graph demonstrating that OX activates BMP signaling in mesenchymal stem cells. The results of an assessment of BMPR1a expression by qPCR are shown.

FIG. 22 is a graph showing the results of a qPCR assessment of BMP-7 and demonstrates that OX activates BMP signaling in mesenchymal stem cells.

FIG. 23 is a photomicrograph of cell cultures showing lipid accumulation in mesenchymal stem cells in conjunction with dorsomorphin as illustrated by Oil Red O staining.

FIG. 24 is a schematic illustration of a proposed model for the role of orexin regulation of brown adipocyte development.

FIG. 25 (a)-(d) is a series of bar graphs demonstrating the induction of BAT activity by peripheral OX injection and the effect of the injection on (a) energy spent, (b) physical activity, (c) energy intake, and (d) oxygen consumption.

FIG. 26 is a bar graph showing the quantification of the gene expression changes in iBAT following injections of OX and isoproterenol.

FIG. 27 demonstrates the prophylactic effect of OX against weight gain. FIG. 27(a) is a graph showing the comparison of the body weights of OX KO with wild-type mice. FIG. 27(b) is a graph showing the comparison of energy intake between wild-type mice injected with vehicle or OX. FIG. 27 (c) is a graph showing the variance of cumulative energy consumed between wild-type and OX mice. FIG. 27 (d) is a graph showing the variance in body weight between the same. FIG. 27 (e) of mice demonstrating the differences in body size between those receiving vehicle or OX. FIG. 270 is a graph demonstrating the effect of OX on fat mass weight. FIG. 27(g) is a graph demonstrating the effect of OX on lean mass weight. FIG. 27(h) and (i) are photomicrographs showing the abdomen and brown fat, respectively, of mice receiving OX and vehicle control.

FIG. 28 demonstrates the effects of OX in conferring resistance to obesity. FIGS. 28(a) and (b) are line graphs showing the energy intake (a) and body weight (b) of mice treated with OX and control mice treated with vehicle over a period of six weeks. FIGS. 28(c), (d), (e), and (f) are photographs showing the abdominal fat (c)-(d) and total white visceral fat (e)-(f) of mice fed a high-fat diet and treated with either vehicle (c), (e) or OX (d), (f). FIGS. (g)-(j) are bar graphs showing physical activity (g), metabolic rate (h), energy expenditure (i), and respiratory quotient (j) of the vehicle- and OX-treated mice. FIGS. 28(k) and (l) show a comparison in iBAT UCP1 expression of vehicle- and OX-treated mice, showing a bar graph of mRNA results (k) and a photograph of protein expression results (l).

FIG. 29 demonstrates the ability of OX to reverse already-acquired obesity without a reduction in calorie consumption. FIG. 29(a) shows a line graph of the growth curves in body weight prior to beginning treatment, and after treatment with either OX or vehicle. FIGS. 29(b) and (c) show bar graphs of average food intake and physical activity over a 24 hour period, respectively, of the control- and OX-treated populations. FIG. 29 (d) is a series of photographs showing the gross differences in abdominal fat in pre-treatment mice and after four weeks of either control of OX treatment. FIG. 29(e) is a photograph showing the livers of control- and OX-treated mice. FIG. 29(f) is a photograph showing the coloring of brown adipose tissue of control- and OX-treated mice. Finally, FIG. 29(f) shows mitotracker staining of iBAT.

FIG. 30 is a schematic showing an overview of the acute control of adipose tissue activity.

FIG. 31 is a schematic showing the β₃- and α₂-adrenergic signaling pathways in mature brown adipocytes.

### DETAILED DESCRIPTION

The present invention is based on the discovery that orexin (OX) is a potent trigger for both brown preadipose tissue differentiation as well as BAT activity and energy expenditure. Therefore, OX may be used confer resistance to diet-induced obesity by controlling weight gain and/or promoting weight loss without the necessity of a reduction in food intake or an increase in physical activity.

### Orexin

OX (also referred to as hypocretin) is a neuropeptide hormone produced by the lateral hypothalamic area (LHA); it regulates sleep-wake cycles, physical activity, and appetite. Consequently, its depletion impacts arousal and diminishes ambulation and feeding. OX also orchestrates temporal changes in expression of early, intermediate, and terminal differentiation markers and activates transcriptional regulators of brown fat leading to lipidogenesis, mitochondrial biogenesis, and uncoupled respiration. It is provided herein that a pharmaceutical composition comprising OX, formulated as described in detail below, increases BAT activity, triggers brown preadipose tissue differentiation, and enhances energy expenditure to combat obesity, even with increased caloric intake.

Two types of OX are known: a major peptide OX-A, which comprises 33 amino acids (approximately 3.5 kDa) and is well conserved in mammalian species, and a minor peptide OX-B, which comprises 28 amino acids (approximately 2.9 kDa) and has a 46% homology with OX-A. These two peptides are the result of proteolytic cleavage of a single precursor protein, 130-131 amino acid prepro-orexin. The human prepro-orexin gene is located on chromosome 17q and consists of only two exons and one intron. After detachment of the N-terminal 33-amino acid residue signal peptide, prepro-orexin (now pro-orexin) is cleaved by prohormone convertases to yield one molecule each of orexin-A and orexin-B. Orexin-A is much more stable than Orexin-B, which explains why its tissue and blood concentrations are markedly higher. Moreover, orexin-A displays higher liposolubility than orexin-B, which makes it, in contrast with orexin-B, blood-brain barrier permeant. The amino acid sequence for orexin-A is as follows: pGlu-Pro-Leu-Pro-Asp-Cys-Cys-Arg-Gin-Lys-Thr-Cys-Ser-Cys-Arg-Leu-Tyr-Glu-Leu-Leu-Flys-Gly-Ala-Gly-Asn-His-Ala-Ala-Gly-Ile-Leu-Thr-Leu (SEQ ID NO.: 1). See Spinazzi et al., Orexins in the Regulation of the Hypothalamic-Pituitary-Adrenal Axis, Pharmacological Reviews, Vol. 58, 46-57, 2006. Unless specifically indicated otherwise, as used herein, orexin (“OX”) refers to orexin-A.

Two cloned orexin receptors OX1R and OX2R are serpentine G-protein-coupled receptors, both of which hind orexins and are coupled to calcium mobilization. The interest of investigators in orexins has focused on narcolepsy, since genetic or experimental alterations of the orexin system are associated with this sleep disorder. However, orexins are not restricted to the hypothalamus and together with their receptors they are expressed in peripheral tissues. For a complete discussion, see Voisin et al., Orexins and their receptors: structural aspects and role in peripheral tissues, Cell. Mol. Life. Sci., Vol. 60(1), 72-87, 2003, which is hereby incorporated by reference in its entirety.

### Brown Adipose Tissue

As described in Cannon and Nedergaard, Brown Adipose Tissue: Function and Physiological Significance. Physiol Rev 84: 277-359, 2004, the function of brown adipose tissue is to transfer energy from food into heat; physiologically, both the heat produced and the resulting decrease in metabolic efficiency can be of significance. Both the acute activity of the tissue, i.e., the heat production, and the recruitment process in the tissue (that results in a higher thermogenic capacity) are under the control of norepinephrine released from sympathetic nerves. In thermoregulatory thermogenesis, brown adipose tissue is essential for classical nonshivering thermogen-esis (this phenomenon does not exist in the absence of functional brown adipose tissue), as well as for the cold acclimation-recruited norepinephrine-induced thermogenesis. Heat production from brown adipose tissue is activated whenever the organism is in need of extra heat, e.g., postnatally, during entry into a febrile state, and during arousal from hibernation, and the rate of thermogenesis is centrally controlled via a pathway initiated in the hypothalamus. Feeding as such also results in activation of brown adipose tissue; a series of diets, apparently all characterized by being low in protein, result in a leptin-dependent recruitment of the tissue; this metaboloregulatory thermogenesis is also under hypothalamic control. When the tissue is active, high amounts of lipids and glucose are combusted in the tissue. The development of brown adipose tissue with its characteristic protein, uncoupling protein-1 (UCP1), was probably determinative for the evolutionary success of mammals, as its thermogenesis enhances neonatal survival and allows for active life even in cold surroundings.

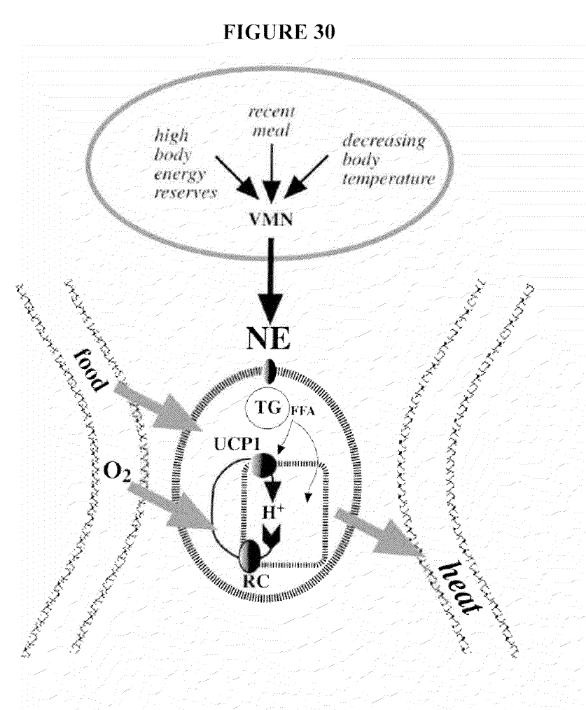

An overview of the acute control of brown adipose tissue activity is shown in FIG. 30. Information on body temperature, feeding status, and body energy reserves is coordinated in the ventromedial hypothalamic nucleus (VMN). When there is reason to increase the rate of food combustion (decrease metabolic efficiency or increase the rate of heat production, a signal is transmitted via the sympathetic nervous system to the individual brown adipocytes. The released transmitter, norepinephrine (NE), initiates triglyceride breakdown in the brown adipocytes, primarily via β₃-adrenergic receptors. The intracellular signal is transmitted via cAMP and protein kinase A, leading to the release from triglycerides (TG) of fatty acids (FFA) that are both the acute substrate for thermogenesis and (in some form) the regulators of the activity of uncoupling protein-1 (UCP1, thermogenin). Combustion of the fatty acids in the respiratory chain (RC) leads to extrusion of H⁺, and UCP1 thus allows for mitochondrial combustion of substrates, uncoupled from the production of ATP, by functionally being (the equivalent of) a H⁺ transporter. The outcome is that an increased fraction of the food and the oxygen available in the blood is taken up by the tissue and combusted therein, leading to an increased heat production. The participation of brown adipose tissue in total energy metabolism is, at least in smaller mammals, very substantial; at “normal” ambient temperatures, nearly one-half of their energy metabolism may be related to brown adipose tissue activity, and in small mammals living in cold environments, the predominant energy utilizer is brown adipose tissue. The capacity of the tissue for the metabolism of the animals alters thus as an effect of environmental conditions: it atrophies when not needed and it becomes recruited when a chronic, high demand is encountered.

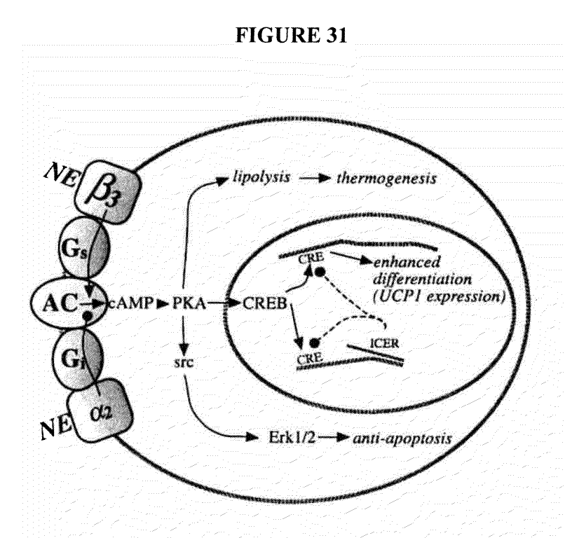

The β₃- and α₂-adrenergic signaling pathways in mature brown adipocytes are shown in FIG. 31. NE, norepinephrine; Gₛ, stimulatory G protein; Gᵢ, inhibitory G protein (dashed lines with solid circles denote inhibition); AC, adenylyl cyclase; PKA, protein kinase A; CREB, CRE-binding protein; CRE, cAMP response element; ICER, inducible cAMP early repressor (it is the resulting protein that inhibits the stimulatory effect of phosphorylated CREB on its own transcription and on that of certain other proteins).

The further β-adrenergic signaling cascade is mediated via adenylyl cyclase activation: the norepinephrine-induced cAMP formation is fully mediated via β₃-receptors in mature brown adipocytes. Correspondingly, all tested β-adrenergic effects, including thermogenesis, can be mimicked by the adenylyl cyclase activator forskolin. It is not fully established which of the 10 adenylyl cyclase isoforms that are responsible for mediating the signal in mature brown adipocytes; several are expressed in brown adipose tissue, and there are functional indications of a change in active adenylyl cyclase isoform during brown adipocyte differentiation. For a complete discussion of the pathway mediating BAT differentiation and formation, see Cannon and Nedergaard.

### Formulations

For clinical use, the compounds of the disclosure are formulated into pharmaceutical formulations for various modes of administration. It will be appreciated that the compounds may be administered together with a physiologically acceptable carrier, excipient, or diluent. The pharmaceutical compositions may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the disclosure may be incorporated into slow release formulations.

The dose level and frequency of dosage of the specific compound will vary depending on a variety of factors including the potency of the specific compound employed, the metabolic stability and length of action of that compound, the subject's age, body weight, general health, sex, diet, mode and time of administration, rate of excretion, drug combination, the severity of the condition to be treated, and the subject undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

Pharmaceutical compositions of the invention can be administered to a subject, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

Methods well known in the art for making formulations are found, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, view York. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated napthalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The amount of active ingredient in the compositions of the invention can be varied. One skilled in the art will appreciate that the exact individual dosages may be adjusted somewhat depending upon a variety of factors, including the protein being administered, the time of administration, the route of administration, the nature of the formulation, the rate of excretion, the nature of the subject's conditions, and the age, weight, health, and gentler of the subject. Generally, dosage levels of between 0.1 mg/kg to 100 mg/kg of body weight are administered daily as a single dose or divided into multiple doses. Desirably, the general daily dosage range is about 0.10, 0.25, 0.50, 0.75, 1, 5, 10, 20, 30, 40, 50, 60, 70, 80, 90, or 100 mg/kg. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

If more than one agent is employed, each agent may be formulated in a variety of ways that are known in the art. Desirably, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. The individually or separately formulated agents can be packaged together or separately, or may be co-formulated.

Generally, when administered to a subject, the timing dosage of any of the therapeutic agent(s) will depend on the nature of the agent, and can readily be determined by one skilled in the art. Each, agent may be administered once or repeatedly over a period of time (e.g., including for the entire lifetime of the subject).

### EXAMPLES

The present methods, thus generally described, will be understood more readily by reference to the following examples, which are provided by way of illustration and are not intended to be limiting of the present methods and kits.

### Example 1

### Histological Evaluation of Brown Adipose Tissue in OX-Null Mice

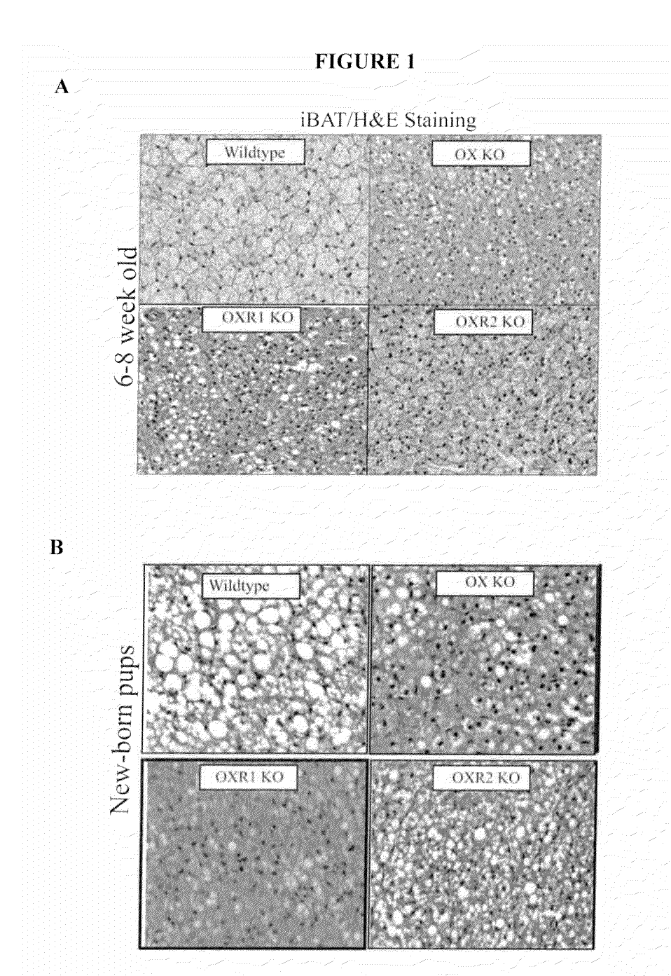

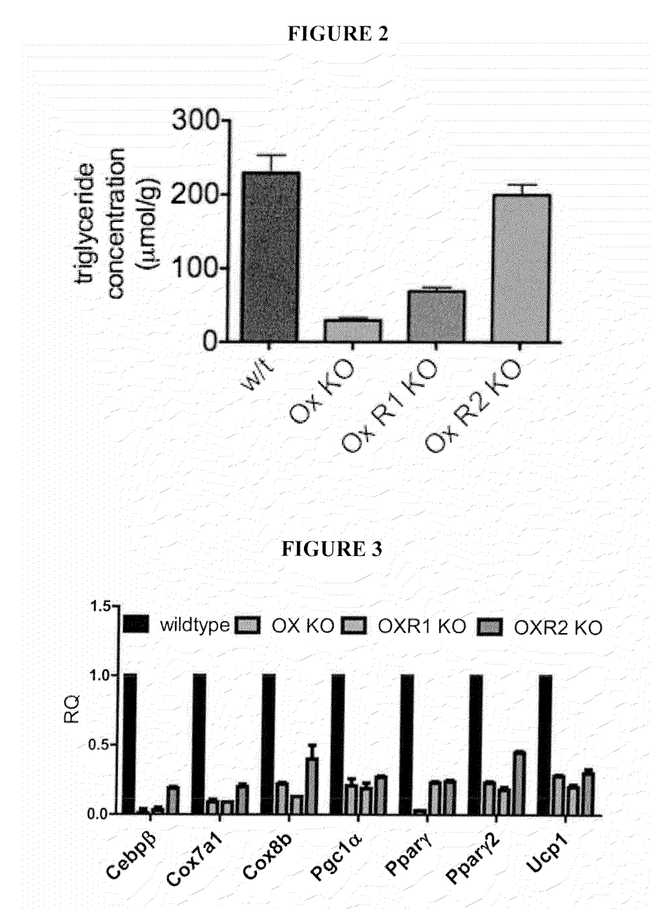

Intrascapular BAT (iBAT) was excised from 6-week-old OX null mice (Jackson Laboratories) and its gross structure and morphology compared with that of wild-type control mice. All mice were housed under standard vivarium conditions with a 12-hour light-dark cycle. iBAT from OX null (OX KO) mice was slightly pale and exhibited abnormal BAT characteristics: H&E staining revealed that brown adipocytes of OX and OXR1 KO mice within the iBAT were depleted of lipid droplets, as reflected by reduced cell size and a thicker cytoplasmic rim (FIG. 1a) and glycerol release (FIG. 2). Most adipocytes contained no lipids, which would appear as optically blank spheres, and remaining cells exhibited small lipid droplets. Nuclei of adjacent brown preadipocytes often appeared in unusually close proximity to one another compared to controls as a consequence of delipidation. H&E staining of iBAT from newborn pups was also conducted with the same parameters, and is shown in FIG. 1b. Lipid content of OX KO and OXR1 KO was reduced. OXR2KO does not impact lipid content of brown adipocytes, but does reduce the size of lipid droplets. It is additionally demonstrated in FIG. 1b that OX and OXR1 knockout, but not OXR2 knockout severely reduces the triglyceride stores as assessed by iBAT glycerol release (FIG. 2). This indicates that OX is indispensable for normal BAT structure.

### Example 2

### OX Signaling and Brown Adipose Tissue Maintenance is Mediated by OXR1

To investigate which of the two OX receptors (OXR1 or OXR2) mediates OX function, morphology of OXR1 null mice was compared with morphology of OXR2 null mice. As discussed above, OXR1 deficiency resulted in brown adipocytes largely devoid of lipids and with a thicker cytoplasmic rim. The impact of OXR2 loss was less severe: lipid content of brown adipocytes did not differ significantly from that seen in the control mice, as shown in FIG. 1a-b. CD31 staining indicated a normal blood supply in both ligand and receptor null mice. Size and morphology of internal organs such as heart and liver in both knockout mice were also equivalent to that seen in wild-type mice. Defects in adipocyte size and lipid content observed in adult knockout mice were also observed in knockout newborns. Lipid droplet size was markedly reduced in iBAT of OXR2 knockout newborns, but the effect was less severe than that observed in OXR1 knockout newborns, as shown in FIG. 1b. OX and either receptor knockout newborn mice were mobile and grossly indistinguishable from wild-type littermate controls, demonstrating that BAT phenotypes observed in newborns are not attributable to differential ambulatory behavior. Further, within mesodermal lineages, histological abnormalities seen in mutant mice appeared specific to adipose tissue (i.e., muscle and connective tissues surrounding BAT in OX, OXR1, and OXR2 knockout mice appeared grossly normal). Finally, loss of either receptor also altered mRNA levels of several important factors regulating BAT function, such as C/3 bp, Cox, Ppar-gamma, Pgc1, and Ucp-1, which indicates that OX signaling is required for the integrity of normal BAT structure and plays an important role in lipid metabolism in brown adipocytes. A summary of mRNA expression of indicated genes in iBAT of wildtype, ligand KO and receptor null mice is provided in FIG. 3. The results demonstrate that expression of the adipogenic regulators (transcription factors) are significantly reduced in OX, OXR1, and OXR2 knockout mice, indicating the BAT production and differentiation is expected to be reduced resulting from a defect in OX signaling. Expression is normalized to 18S RNA.

### Example 3

### Orexin Induces Brown Fat Programming and Differentiation of C3H10T1/2 Mesenchymal Stem Cells

OXR1 expression was confirmed in the undifferentiated mesenchymal stem cell line C3H10T1/2 cells (ATCC). Cells were grown to 50-70% confluence in high glucose DMEM supplemented with 10% FBS and differentiated in standard induction media supplemented with 100 nM human orexin A (cat. No. 24470, Anaspec), vehicle, or recombinant human BMP-7 (cat. No. 4579, BioVision, a potent inducer of RAT differentiation) for three days, at which time cells reached 100% confluence. Cells were then incubated in adipogenic media for 7 days in the absence of and BMP-7. Cells were then stained with Oil Red O according to the following protocol. Cells were washed twice with phosphate-buffered saline (PBS), fixed with 10% buffered formalin for 1 hour at room temperature, washed twice in PBS, stained for 30 minutes at room temperature with a filtered Oil red O (Sigma) solution (0.5% Oil red O in isopropyl alcohol), washed twice with PBS, and stored in PBS for visualization under the inverted microscope (Olympus).

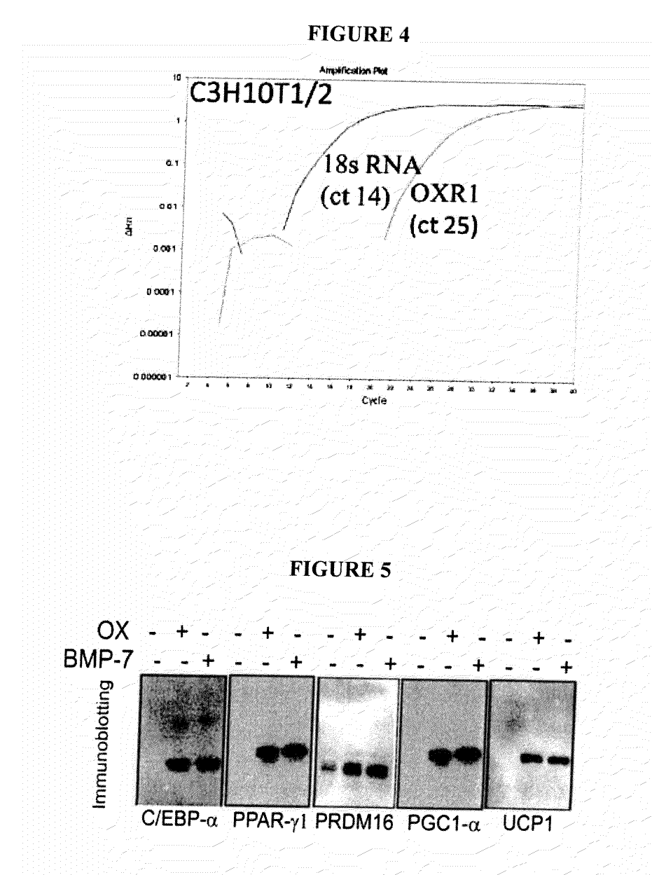

Following OX treatment, greater than 90% of cells differentiated from an elongated fibroblastic morphology to a spherical one typical of differentiated fat cells. OXR1 expression in the differentiated mesenchymal cells was done using RT-PCR analysis of the OXR1 mRNA. FIG. 4 provides the Ct value OXR1 and the Ct value for 18S RNA is shown for comparison.

The rate and extent of cytoplasmic triglyceride accumulation following OX treatment was comparable to that seen following treatment with BMP-7. To determine triglyceride content, BAT was homogenized in 1 ml of saline solution and Triglycerides Reagent Kit (Pointe Scientific) was used to determine triglyceride concentration in the tissue.

Protein extracts from the treated mesenchymal cells was used to assess the expression of early adipogenic transcription factors that are known to function in adipogenesis. Specifically, immunoblotting was used to assess the expression of C/ebp-alpha, Ppar-γ1, Prdm16, Pgc1-alpha, and Ucp1 in the differentiated mesenchymal stem cells. OX treatment induced the expression of these adipogenic transcription factors in the cultured mesenchymal cells to levels comparable to that induced by BMP-7 (FIG. 5), demonstrating that OX can induce BAT differentiation.

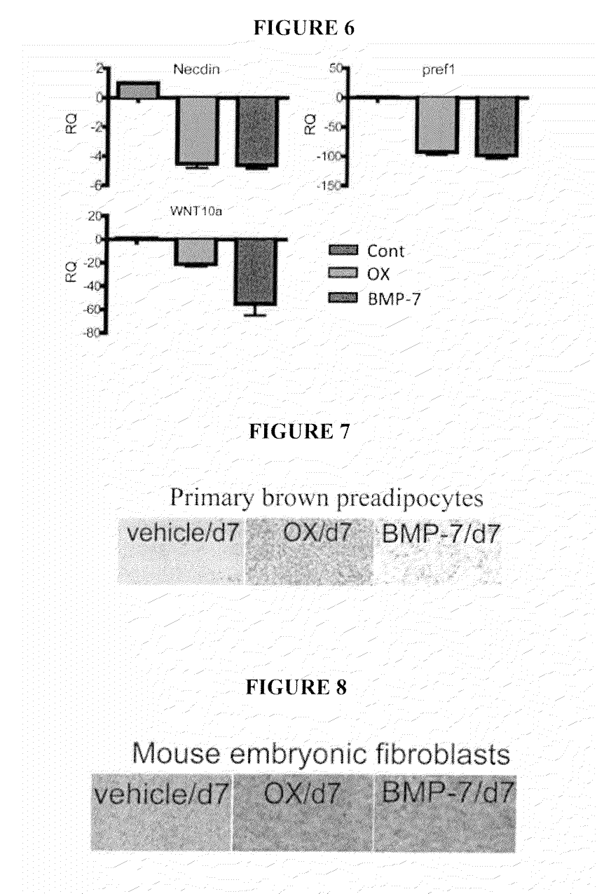

The expression of several adipogenic inhibitors was assessed in the differentiated mesenchymal stem cells and control cells by qRT-PCR. Undifferentiated C3H10T1/2 cells abundantly express Necdin, preadpocyte factor-1 (Pref-1), and Wnt10a, all of which inhibit early adipogenic events. Preadipocytes must counteract an adipogenic block imposed by these factors in order to differentiate. OX-treated cells, in contrast, showed suppression of mRNAs encoding inhibitory factors Necdin. Preadipocyte factor-1, and Wnt10a, most notably Pref-1, whose expression decreased by two orders of magnitude following OX treatment. FIG. 6 illustrates results from a PCR analysis of adipogenic inhibitors in C3H10T1/2 cells treated with BMP-7 or OX for 3 days followed by adipogenic induction for 7 days. Data are expressed as arbitrary units after normalization to 18S RNA.

The cultured mesenchymal stem cells were assessed for the expression of a variety of regulators of adipogenesis and mitochondrial function, qRT-PCR was performed as follows: RNA was isolated using Trizol lysis reagent (Qiagen) and purified by RNeasy Mini columns (Qiagen), cDNA was produced using an RT-PCR kit (Applied Biosystems) and primers synthesized by Integrated DNA Technologies, and PCR reactions were run in duplicate for each sample and quantified in the ABI Prism 7000 Sequence Detection System (Applied Biosystems) The expression of each RNA was normalized to the 18S RNA level. A listing of primers is provided in Table 1.

TABLE 1

| Mouse Primers | RefSeq (mRNA) | | SEQ ID |
|---|---|---|---|
| Gene Name | Accession No. | Primer Sequences | NO.: |
| BMPR1a | NM_009758 | tggctgtctgtatagttgctatgat | 2 |
| | | tgcttgagatactcttacaataatgct | |
| BMP7 | NM_007557 | cgagaccttccagatcacagt | 3 |
| | | cagcaagaagaggtccgact | |
| PGC1β | NM_133249 | tgccacaacccaaccagtctca | 4 |
| | | agcagtctccagcagcccaaag | |
| PRDM16 | NM_027504 | acaggcaggctaagaaccag | 5 |
| | | cgtggagaggagtgtcttcag | |
| SCD1 | NM_009127.4 | ttccctcctgcaagctctac | 6 |
| | | cagagcgctggtcatgtagt | |
| COX7a1 | NM_009944 | ctgaggacgcaaaatgagg | 7 |
| | | tggcttctggtagatgagctaaa | |
| COX8b | NM_12909344 | ccagccaaaactcccactt | 8 |
| | | gaaccatgaagccaacgac | |
| SIRT3 | NM_022433 | tcctctgaaaccggatgg | 9 |
| | | tcccacacagagggatatgg | |
| TFAM | NM_009360 | caaaggatgattcggctcag | 10 |
| | | aagctgaatatatgcctgcttttc | |
| PPARγ1 | NM_001127330.1 | aaacaacgcaacgtggaga | 11 |
| | | gcggtcattgtcactggtc | |
| PPARγ2 | NM_011146.3 | gaaagacaacggacaaatcacc | 12 |
| | | gggggtgatatgtttgaacttg | |
| UCP1 | NM_009463 | ggcctctacgactcagtcca | 13 |
| | | taagccggctgagatcttgt | |
| CEBPα | NM_007678 | aaacaacgcaacgtggaga | 14 |
| | | gcggtcattgtcactggtc | |
| CEBPβ | NM_009883 | tgatgcaatccggatcaa | 15 |
| | | cacgtgtgttgcgtcagtc | |
| PGC1α | NM_008904 | cggaaatcatatccaaccag | 16 |
| | | Tgaggaccgctagcaagtttg | |
| OXR1 | NM_198959 | aaggtccaggttccagca | 17 |
| | | ggtatcattattagcaagccctgt | |
| Adam15(1) | NM_00103772 | agcacaggaatgtcgaagaaa | 18 |
| | | ttgagctgggtcatgcagt | |
| FOXC2 | NM_013519 | cggctaggactggacaactc | 19 |
| | | ctgacagctcgcattgctc | |
| ZFP423 | XM_001000774 | cgcctgggattcctctgt | 20 |
| | | ctggttttccgatcacactct | |
| NECDIN | NM_010882 | aacaaccgtatgcccatga | 21 |
| | | acatagatgaggctcaggat | |
| PREF-1 | NM_010052 | cgggaaattctgcgaaatag | 22 |
| | | tgtgcaggagcattcgtact | |
| WNT10a | NM_009518 | ggcgctcctgttcttccta | 23 |
| | | gtcgttgggtgctgacct | |
| Human Primers | RefSeq (mRNA) | | SEQ ID |
| Gene Name | Accession No. | Primer Sequence | NO.: |
| UCP1 | NM_021833.4 | ctggacacggccaaagtc | 24 |
| | | gacacctttatacctaataacactgg | |
| PPARγ1 | NM_138712.3 | gacaggaaagacaacagacaaatc | 25 |
| | | ggggtgatgtgtttgaacttg | |
| PPARγ2 | NM_015869.4 | tccatgctgttatgggtgaa | 26 |
| | | tgtgtcaaccatggtcatttc | |
| PRDM16 | NM_022114.2 | tacactgtgcaggcaggcta | 27 |
| | | gtgtggagaggagtgtcttcg | |
| FOXC2 | NM_005251 | ggggacctgaaccacctc | 28 |
| | | aacatctcccgcacgttg | |
| Cox7a1 | NM_001864 | gacaatgacgctgtgtctgg | 29 |
| | | cccaggcttcttggtcttaat | |
| SIRT3 | NM_001017524 | cttgtgcagcgggaaact | 30 |
| | | tcctatgttaccatttattgtgtgg | |
| NRF1 | NM_001040110 | ccatctggtggcctgaag | 31 |
| | | gtagtgcctgggtccatga | |
| OXR1 | NM_001525.2 | tacgcctgcttcaccttctc | 32 |
| | | taaactgctcccggaatttg | |

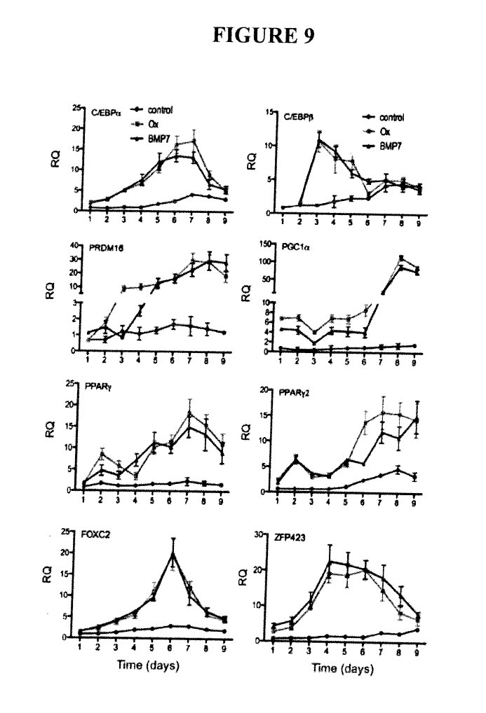

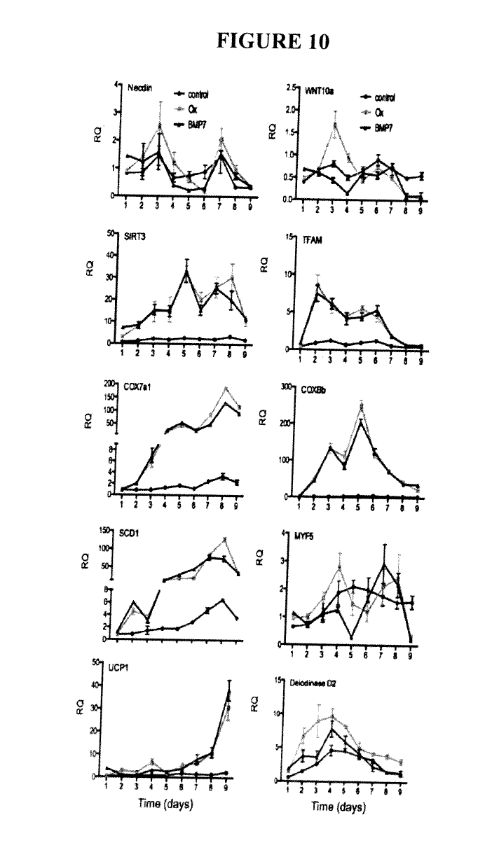

Important adipogenic regulators such as C/ebp. Prdml6, Ppar-gamma, Foxc2, and Zfp423 were significantly increased prior to suppression of adipogenic inhibitors, as demonstrated in FIG. 9. These results demonstrate that OX-induced brown fat lineage commitment in this system is insensitive to adipogenic inhibitors. Mitochondiral transcription factor (Tfam), cytochrome oxidase (Cox7a, Cox8b) and deiodinase-2 expression were elevated before exposure to adipogenic media, as shown in FIG. 10. These results demonstrate that OX induces expression of genes involved in mitochondrial biogenesis and function and does not require adipogenic conditions. Expression of stearoyl-CoA desaturase (Scd), an enzyme catalyzing the rate-limiting step in lipid biosynthesis, was also elevated over 100-fold during differentiation. mRNAs encoding other markers fatty acid oxidation such as Lp1, Sirt3, Adam15-1 and Adam15-2, and Adipor1 were enriched in differentiated mesenchymal stem cells following OX treatment (FIG. 11). Importantly, OX induced Ucp-1 and deiodinase type 2 (Dio1) mRNA expression, indicating that OX can induce transcriptional changes relevant to thermogenesis.

### Example 4

### Orexin Induces a Brown Fat Program

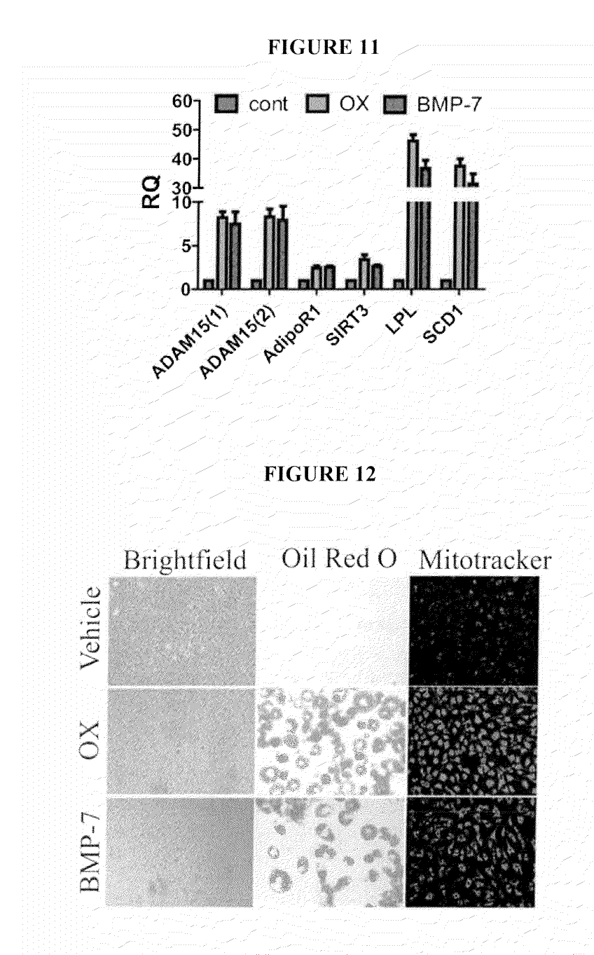

Based on the foregoing studies, three features distinguish BAT from WAT: the appearance of multilocular oil droplets, mitochondrial enrichment, and Ucp-1 expression. To further investigate whether OX induces a brown fat differentiation program in mesenchymal stem cells, C3H10T1/2 cells were treated with either vehicle or OX, as above, and stained with Oil Red O on the final day of differentiation. Tissues were fixed in 10% formalin and were paraffin-embedded. Multiple sections were prepared and stained with haematoxylin and eosin for general morphological observation. BMP-7 pretreated cells served as a reference, as delineated in FIG. 12. Following OX treatment, greater than 90% of cells assumed the spherical morphology typical of differentiated fat cells. OX treatment loaded adipocytes with multiple small cytoplasmic oil droplets and induced extensive mitochondrial biogenesis as determined by MitoTracker (FIG. 12). For mitochondrial staining using MitoTracker® Red FM, cells were incubated with pre-warmed medium containing the MitoTracker probe at a working concentration of 250 nM, Cells were then fixed in 4% formaldehyde and observed using fluorescent microscope. Expression of genes involved in mitochondrial biogenesis and function, such as Pgc1-a, Pgc1-p, C/ebp-a, Prdm16, Pgc-1, nuclear respiratory factor 1 (Nrf1), Tfam, and cytochrome c, were markedly elevated.

### Example 5

### Orexin-Induced Respiration is Uncoupled from ATP Synthesis

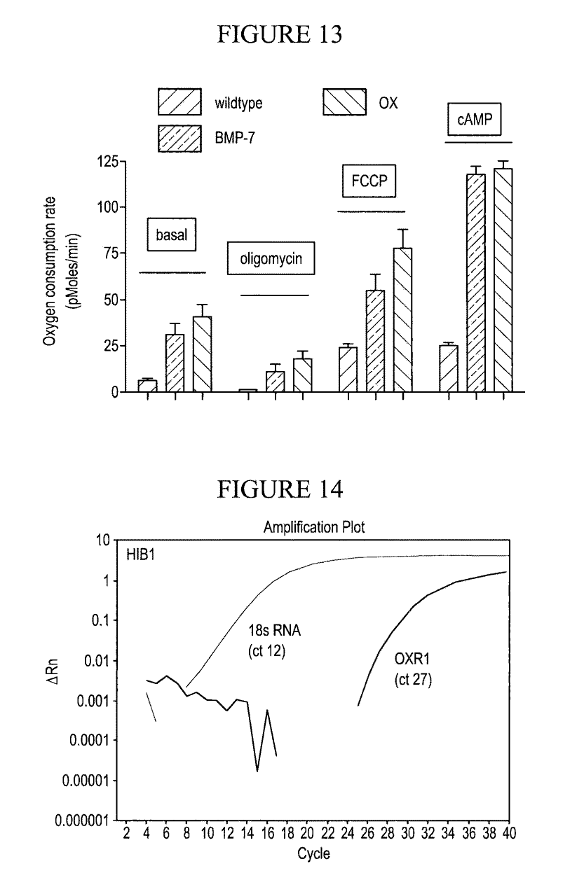

In view of the increased mitochondrial biogenesis observed following OX treatment, the respiratory activity in cultured mesenchymal stem cells was assessed. OX-treated cells displayed 15-fold higher oxygen consumption (FIG. 13; basal conditions).

To determine whether the increased respiration was uncoupled from ATP synthesis, oligomycin-insensitive respiration was first assayed as a measure of uncoupled respiration. Oligomycin inhibits F1 ATP synthetase to suppress only oxidative phosphorylation-associated respiration. As a result, all residual respiration is due to uncoupling. In the presence of oligomycin, OX-treated cells or BMP-7-treated cells efficiently consumed oxygen, reflecting uncoupled respiration (FIG. 13; oligomycin). More than half of OX-induced respiration was uncoupled from ATP synthesis, an attribute of brown fat. In contrast, oligomycin completely suppressed respiration in unstimulated differentiated indicating that in the absence of OX, nearly all cellular respiration is coupled to ATP synthesis.

In the presence of FCCP, an uncouples used to maximize respiratory activity, oxygen consumption of unstimulated, differentiated cells increased 6-fold (FIG. 13). Oxygen consumption rate, uncoupled respiration and expression of UCP1 were further stimulated by cAMP when cells were cultured in the presence of OX, suggesting that differentiated C3H10T1/2 cells resemble BAT and can execute a thermogenic program. That FCCP had a lesser effect in OX- and BMP-7-treated cells demonstrates that basal electron transport activity of these cells is near maximal. Together, these data confirm that OX is a potent inducer of brown fat adipogenesis in mesenchymal stem cells.

### Example 6

### Orexin's Role in Differentiation of Hibernoma HIB1) Brown Preadipocytes

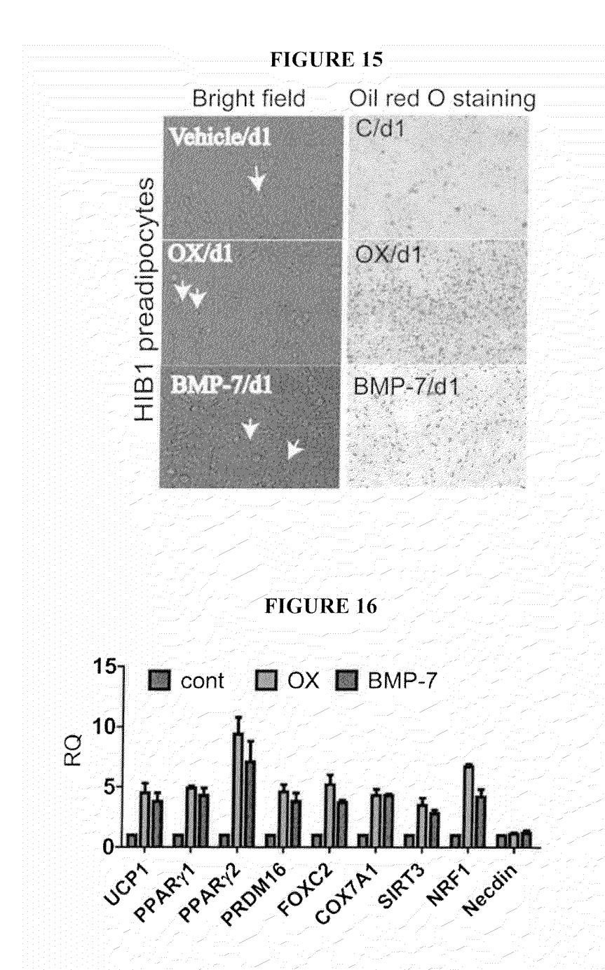

To further investigate the role of OX in BAT differentiation, the effect of OX on the preadipocyte cell line HIB1 was evaluated. It was found that HIB1 cells express moderate levels of OXR1 (FIG. 14). In the absence of the standard induction cocktail, (IBMX, thiazolidone, and indomethacin), OX treatment of HIB1 cells for just 24 hours induced extensive lipid accumulation and mitochondrial biogenesis (FIG. 15). Next, HIB1 cells were treated for 3 days with Orexin A or BMP7 and differentiated in the absence of induction medium for a further 7-10 days. Expression of genes functioning in lipid metabolism and mitochondrial biogenesis was elevated while the anti-adipogenic factors Nectin, Pref1, and Wnt10 were expressed at very low levels (FIG. 16). That neither OX nor BMP-7 treatment significantly altered expression of any of these factors during the entire course of differentiation demonstrates that they are not key regulators of HIB1 differentiation.

### Example 7

### Orexin Induces Differentiation of Primary Brown Adipocytes

To assess differentiation of primary brown adipocytes, iBAT preadipocytes were isolated from 1-day-old mice and then differentiated in the presence of OX. Differentiation was confirmed by Oil Red O staining which visualizes lipid accumulation (FIG. 7). OX-treated cells displayed robust adipogenesis within 7 days accompanied by a marked increase in expression of BAT-specific transcriptional regulators and thermogenic proteins. Taken together. OX activates a full program of brown fat adipogenesis by suppressing adipogenic inhibitors, including BAT regulators, elevating mitochondrial biogenesis and oxygen consumption, and inducing uncoupled respiration.

Mouse embryonic fibroblasts (MEFs) resemble mesenchymal cells in their ability to differentiate into various mesenchymal lineages. To determine whether OX triggers commitment of embryonic fibroblasts to a BAT lineage, MEFs isolated at PI3.5 were exposed to a differentiation protocol involving a 3-day treatment with Orexin A and BMP-7, and differentiation in the absence of the standard induction cocktail of IBMX, thiazolidone, and indomethacin for a further 7-10 days. OX-treated MEFs also adopted a BAT phenotype, confirmed by Oil Red O staining (FIG. 8). OX-treated cells upregulated Prdm16, Pparγ-1, Pparγ-2, Sirt3, and Tfam mRNAs, leading to considerable lipidogenesis, as shown in FIG. 9, and increased expression of mRNAs encoding the thermogenic protein Upc1.

### Example 8

### Endogenous OX Drives BAT Differentiation

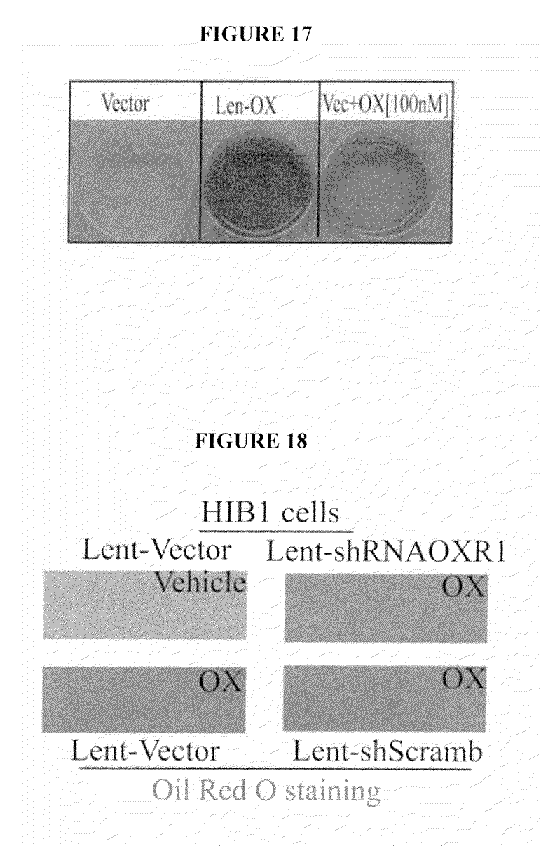

HIB1 cells were stably transfected with lentivirus over-expressing orexin (Len-OX) and compared to vector controls in the absence or presence of exogenous OX (100 nM), Cells were cultured for 7 days in the absence of differentiation medium and stained with Oil Red O. By day 6 of adipogenic differentiation, orexin-expressing cells had undergone normal BAT differentiation as scored by lipid accumulation. The extent of lipid accumulation in orexin-expressing cells was significantly greater than that seen in cells treated with exogenous OX, as shown in FIG. 17.

### Example 9

### Consequences of OXR1 Depletion

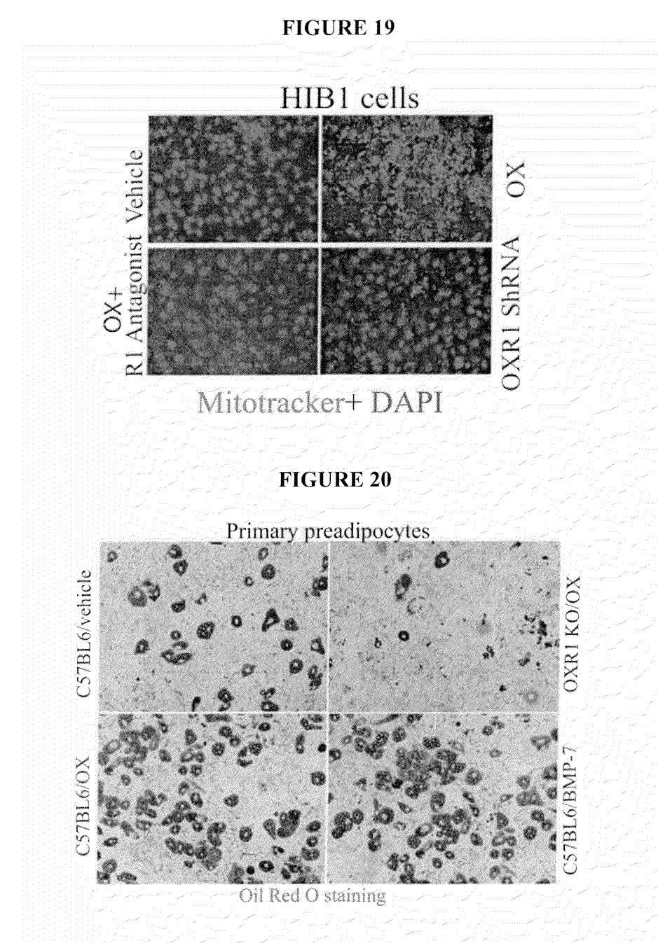

Brown fat morphological defects seen in both adult and newborn OXR1 KO mice were similar to those observed in OX KO mice. Furthermore, OX triggered brown fat differentiation in C3H10T1/2 cells, which express only OXR1, demonstrating that OX couples to OXR1 to induce differentiation. To examine the cellular and molecular consequences of OXR1 depletion, lentiviral vectors were used to express a short hairpin (sh) shRNA targeting OXR1 (shOXR1; Open Biosystems, Inc., catalog no. RMM4431-98766481) or a scrambled shRNA control (Open Biosystems, Inc., catalog no. RHS4346) in HIB1 mesenchymal cells. OXR1 mRNA was virtually undetectable in cultures expressing the shOXR1 construct. After 5 days of differentiation, cultures expressing the scrambled shRNA control had undergone brown fat differentiation as scored by Oil Red O staining and shown in FIG. 18. By contrast, shOXR1-expressing cultures did not differentiation and primarily contained fibroblastic cells. OXR1 depletion, either by lentiviral treatment with shRNA targeting OXR1 or by pharmacological inhibitition using SB408124, 1 uM, blunted OX-dependent mitochondrial biogenesis, as shown in FIG. 19, wherein mitochondrial (red) and nuclear (blue, DAPI) staining after OX treatment for 5 days is shown. Treatment of HIB1 cells with OXR1 selective antagonist SB408124 also blunted orexin-induced mitochondrial biogenesis. Accordingly, OXR1 knockdown ablated expression of brown fat-selective genes, such as Pgc1-alpha, Pgc-1β, Pparg-γ1, Prdm16 and C/ebp-alpha, Cox-8b, Ucp1, Dio2, and Cidea. These results demonstrate that OX couples to OXR1 to mediate BAT differentiation.

To investigate whether lack of OXR1 impaired BAT differentiation potential, primary brown preadipocytes from OXR1 knockout mice were isolated and differentiated. Generation of wild-type primary brown preadipocyte cell lines was derived from newborn wild-type mice as described previously by Klein et al. (Bioessays, 24: 382-388, 2002), which is hereby incorporated by reference in its entirety. Brown preadipocytes isolated from C57BL-6 mice served as positive control. All cell lines were maintained in Dulbecco's modified Earle's medium (DMEM), high glucose, supplemented with 10% FBS at 37° C. in a 5% CO₂ environment. To induce adipogenesis, the cells were treated for 3 days with either BMP7 or 100 nM OX, at which time they were confluent. Cells were then incubated in adipogenic medium containing 0.125 mM indomethaein, 5 mM dexamethazone, and 0.5 uM 3-isobutyl-1-methyxanthine (IBMX) supplemented by 20 nM insulin as described by Tseng et al., (Nature, 454: 1000-1004, 2008) which is hereby incorporated by reference in its entirety. As shown in FIG. 20, wild-type preadipocytes differentiated with low efficiency, while OX or BMP-7 treatment enhanced differentiation based on lipid accumulation. In contrast, brown preadipocytes from OXR1-null mice showed little differentiation capacity, even in the presence of OX (FIG. 20), suggesting that OXR1 is indispensable for BAT differentiation and activity.

### Example 10

### Orexin Signaling Induces Smad 1/5/8 Phosphorylation

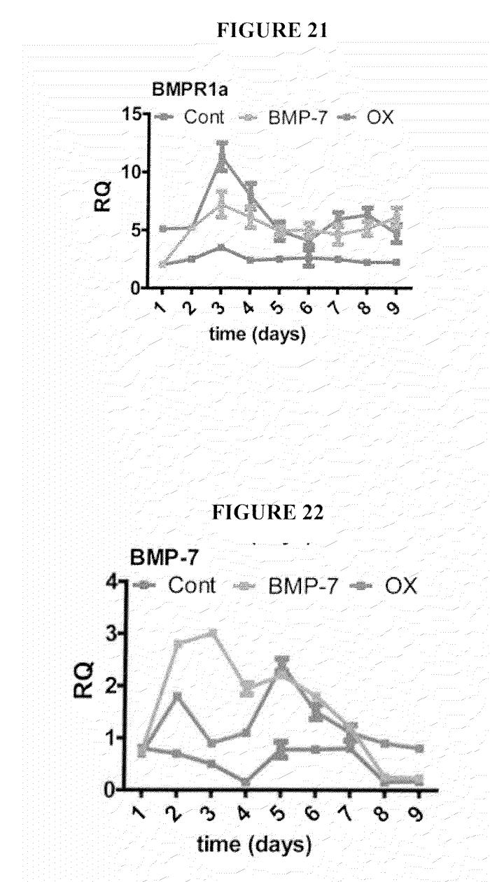

Bone morphogenic proteins, which are members of the TGF-β superfamily, control critical steps in development and differentiation and are important regulators of both WAT and BAT adipogenesis. BMP-2 and -4 induce white fat adipogenesis, while BMP-7 enhances brown fat traits. BMP-7 functions through interacting with BMP receptors, which mediate Smad 1/5/8 phosphorylation, to stimulate brown fat adipogenesis. OX and BMP-7 treatments have almost identical effects on gene expression in mesenchymal stem cells, MEFs, and preadipocytes which demonstrates that OX's effects are relayed by BMP signaling. To demonstrate that OX signaling induces Smad 1/5/8 phosphorylation, C3H10T1/2 mesenchymal stem cells were treated with 100 nM of OX for 3 days and subjected to the differentiation protocol as described above. OX treatment induced BMP-receptor 1A (Bmp1a) and BMP-7 mRNA expression which illustrate the qPCR results, concomitant with Smad 1/5/8 Phosphorylation (FIGS. 21 and 22).

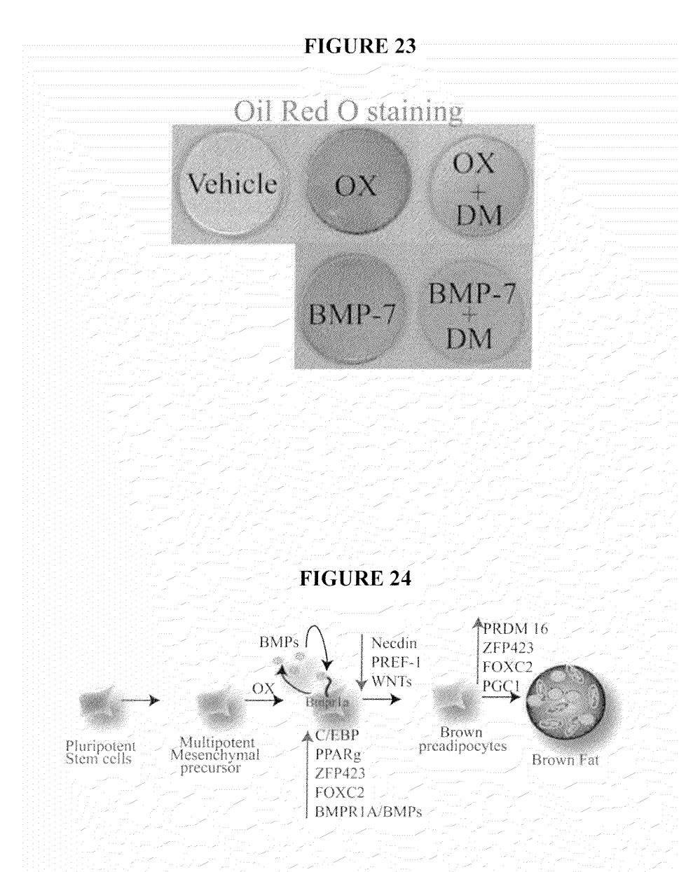

To determine whether OX-triggered adipogenesis requires Bmpr1a, differentiation of mesenchymal stem cells was assessed in the presence of 2 uM dorsomorphin, a selective inhibitor of BMP type I receptors. BMP-7 served as the positive control. Dorsomorphin treatment blunted both OX- and BMP-7-induced brown fat differentiations, as demonstrated by Oil Red O staining for lipid accumulation in cells (FIG. 23). Together, these data demonstrate that OX employs the Bmpr1a receptor to initiate a downstream response and support a model in which OX induces expression of BMP-7, which when secreted, binds to BMP receptors and induces Smad 1/5/8 phosphorylation to drive brown fat adipogenesis as shown in FIG. 24.

### Example 11

### Orexin Enhances BAT Function and Energy Expenditure in Vivo

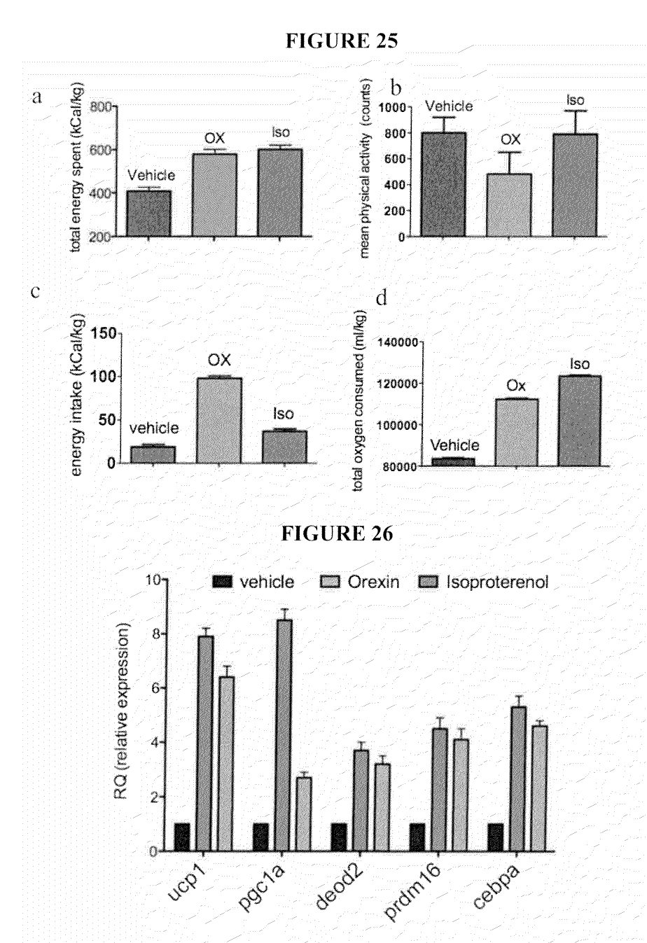

One dose of 30 mg kg⁻¹ OX was administered intraperitoneally in a single dose to 6-8 week C57BL/6 mice, and metabolic rates and energy expenditure of those mice were then compared to 1 mg kg⁻¹ isoproterenol- or vehicle (PBS) control-injected mice. Isoproterenol is a beta-sympathomimetic and serves as a reference for Ucp1 expression and brown fat activity. The results of the comparison of physical activity, energy intake, and oxygen consumption are shown in FIG. 25. Metabolic rates were measured by indirect calorimetry using the Comprehensive Lab Animal Monitoring System (CLAMS, Columbus Instruments). Food and water were available ad libitum. Mice were acclimatized to individual cages for 24 hours prior to recording, and then underwent 24 hours of monitoring.

The single OX injection induced 23-25% increase in whole-body energy expenditure, despite decreased physical activity and increased food consumption. OX injection also stimulated oxygen consumption, indicating increased metabolic rates. Increased energy expenditure was positively correlated with iBAT lipolysis, as evident from depletion of fat droplets. The extent of lipolysis in OX-injected mice was comparable to that induced by isoproterenol. Gene expression analysis revealed that OX induced prdm16, Pgc1-alpha, C/ebp-alpha, Dio2, and Ucp-1 in BAT (see FIG. 26), supporting induction of brown fat activity. As a consequence, core body temperature was elevated by OX injection.

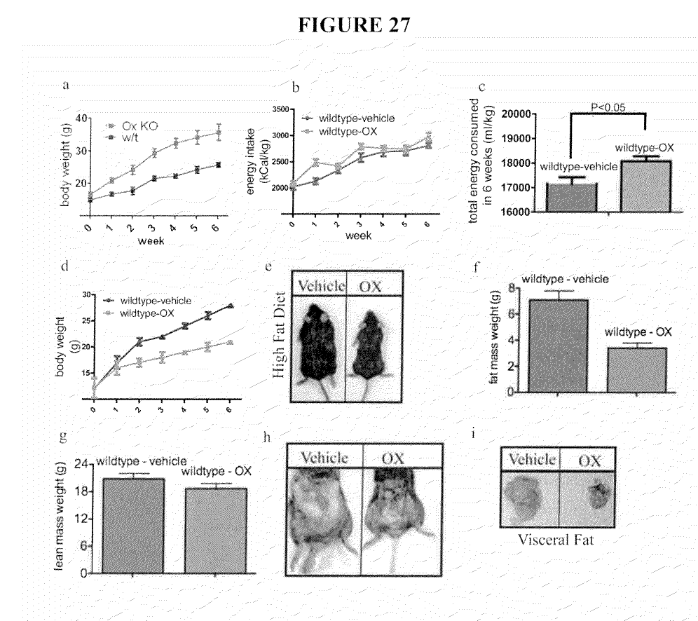

To confirm these findings and investigate the role for OX in energy metabolism and to prevent obesity, wild-type C57BL6 mice were fed a high fat diet (HFD) for six weeks. During this period, half of the mice received OX intraperitoneally once per day, while the other half was injected with saline. Food intake and body weight of both groups were monitored weekly. OX-treated mice ate more, resisted weight gain, were visibly lean, and accumulated less fat. In contrast, control mice were approximately 35% heavier, displayed 3.5 times more abdominal obesity, and accumulated twice as much body fat. Fat and lean mass were determined by subjecting mice to nuclear magnetic resonance (NMR) (Bruker, The Woodlands, Tex., United States) following a four-hour fast. OX therapy had no impact on either the lean mass or total fluid content. FIG. 27 illustrates these findings. Weekly energy intake, cumulative energy consumed, body weight gain over six weeks, and differences in body size between the two groups of mice are depicted. FIG. 27 further demonstrates that OX therapy reduces fat mass weight, but not lean mass weight. FIG. 27a illustrates body weight, FIG. 27b shows energy intake in kCal/kg, FIG. 27c shows cumulative energy consumed, FIG. 27d shows body weight gain over 6 weeks, FIG. 27e shows differences in body size, FIG. 27f demonstrates that orexin therapy reduces fat mass weight, FIG. 27g shows that orexin therapy does not reduce lean mass weight, FIG. 27h illustrates the abdominal fat, and FIG. 27i illustrates total white visceral fat tissue following 6 weeks of OX therapy. Table 2 further shows the NMR results as a quantitative measurement of fat mass, lean mass, lean mass to fat mass ratio, percent body fat and total body water are shown (n=4).

TABLE 2

| | | | Lean mass- | | |
|---|---|---|---|---|---|
| | | | fat mass | % | Fluid |
| | Fat mass | Lean mass | ratio | body fat | content |
| Wildtype | 7.1 ± 0.9 | 20.8 ± 1.2 | 2.9 ± 0.4 | 25 ± 1.3 | 8.4 ± 1.6 |
| Vehicle | | | | | |
| Wildtype | 3.4 ± 0.4 | 18.7 ± 1.1 | 5.5 ± 0.3 | 15 ± 1.1 | 7.7 ± 1.5 |
| OX | | | | | |

### Example 12

### Orexin Induces Obesity Resistance in Wt Mice without Inducing Anorexia or Requiring Physical Activity

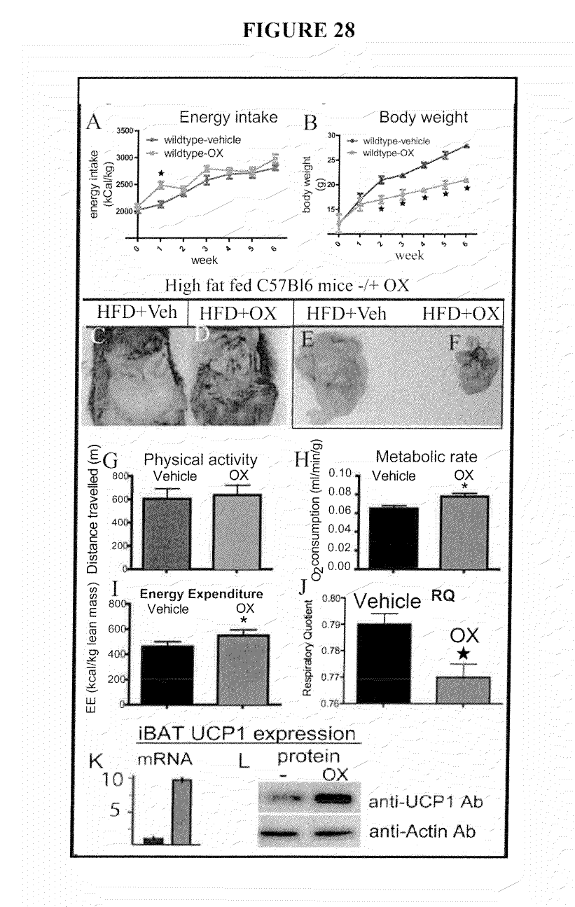

To confirm that OX prevents obesity under conditions of caloric excess, a high fat diet (HFD) was fed to WtB6 mice for six weeks. Mice received two weekly OX or PBS injections (n=6/group). Food intake and body weight of both groups were monitored weekly. Consistent with its appetite inducing effect, OX-treated mice ate significantly more during the first week, as shown in FIG. 28A. Food intake thereafter, was comparable between the groups, indicating that chronically injected OX does not increase calorie intake. Cumulative food intake over the 6-week period was not significantly different between the OX-treated and vehicle-treated populations. OX-injected animals, however, resisted weight gain, which became apparent during the second wk of therapy (FIG. 28B). At the conclusion of the treatment period, OX-treated mice were visibly lean (FIG. 28C, D) and weighed 7 g±1.2 g less than the control group (FIG. 28B, Table 3). Body composition analysis at the end of the study suggested that whole body fat mass was reduced 50% in the OX-injected group. Complete results are shown in Table 1, below. Vehicle-injected animals displayed 25% body fat, which was reduced to 15% in the experimental group. Lean mass or fluid content was not significantly different between the groups. Total visceral fat was reduced by more than 60%. Visceral fat in the OX-injected group was noticeably darker in color relative to control tissue (FIG. 28E, F). These results demonstrate that weight gain may be controlled without reducing calorie intake.

To determine whether the observed anti-obesity effect of systemically injected OX was due to an increase in physical activity, the physical activities of high-fat fed wild-type B6 mice receiving two-weekly injections of either OX or vehicle (PBS) were observed for two-weeks using an infrared monitoring system. Surprisingly, as shown in FIG. 280, the OX-injected group was not more physically active. Calorimetric measurements indicated that OX-injected group consumed 15% more 07 than the control group (FIG. 28H). The OX-injected group showed a 19% increase in twenty-Tour hour energy expenditure (FIG. 28I) relative to control mice in both resting and active phase (not shown), suggesting stimulation of the basal metabolic rate. Further, the respiratory quotient of OX-injected group was 10% lower than the vehicle injected group, indicating higher fat oxidation capacity (FIG. 28J). These observations demonstrate that weight loss is triggered by stimulation of other components of energy expenditure. To determine whether systemic OX injection induces BAT activity, mice with OX (10 mg/kg) or PBS were compared, using relative UCP-1 expression as an indicator for BAT activity 24-hours post-injection. OX-injected mice expressed higher UCP1 levels than controls, as shown in FIGS. 28K and 28L. These results demonstrate that acute appetite inducing effects of OX is temporary, that systemic OX induces catabolic effects by driving Ucp1-dependent thermogenesis, and that systemic OX-therapy protects against diet-induced obesity, an effect that does not depend on anorexia or physical activity.

TABLE 3

| | | | Lean mass- | | |
|---|---|---|---|---|---|
| | | | fat mass | % | Fluid |
| | Fat mass | Lean mass | ratio | body fat | content |
| Wildtype | 7.1 ± 0.9 | 20.8 ± 1.2 | 2.9 ± 0.4 | 25 ± 1.3 | 8.4 ± 1.6 |
| Vehicle | | | | | |
| Wildtype | 3.4 ± 0.4 | 18.7 ± 1.1 | 5.5 ± 0.3 | 15 ± 1.1 | 7.7 ± 1.5 |
| OX | | | | | |

### Example 13

### Orexin Reverses Diet-Induced Obesity in WtB6 Mice without Altering Dietary and Physical Activity Behaviors

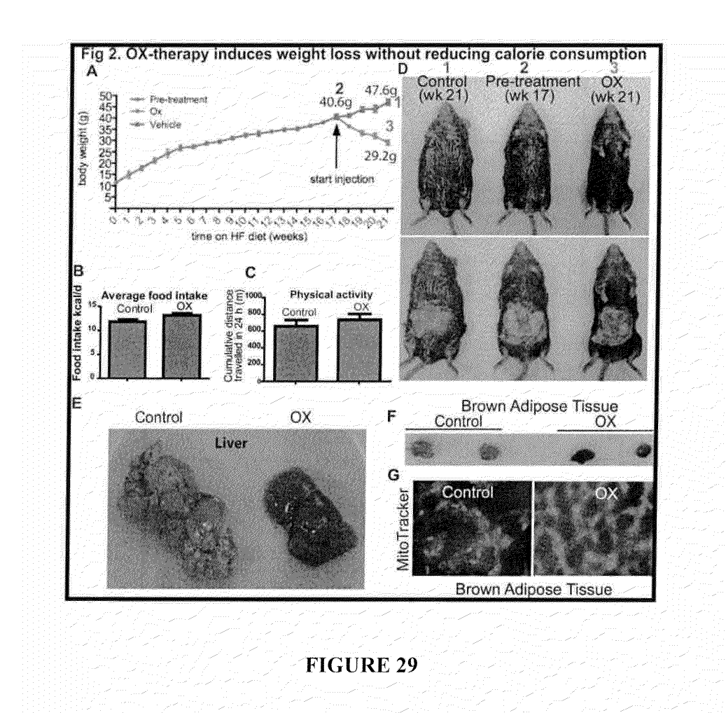

To confirm that systemic OX therapy induces weight loss in obese mice, wild type B6 mice were fed a HFD for 17 weeks and treated either with OX (10 mg/kg) or PBS vehicle twice weekly for 4 wks (n=6 mice/group). The body weight of the control population increased considerably over the 4 weeks, as shown in FIG. 29A, and the mice gained 5 g on average. The OX-injected group began losing body weight from week 1. At the end of the 4-week treatment regimen, the OX-injected population appeared considerably leaner than the vehicle-injected controls, and the former had lost about 10 g body weight, as shown in FIG. 29A. Food intake did not differ significantly between groups, demonstrating that hypophagia does not underlie weight loss (see FIG. 29B). OX-injected mice gained less weight per gram of food consumed, reflecting decreased metabolic efficiency (not shown), and suggesting that physiological mechanisms relevant to energy expenditure might play a role in OX-dependent weight-loss. Calorimetric studies revealed that the OX-treated group expended 17% more energy and consumed 13% more oxygen, indicating higher metabolic rate compared to the control group. Enhancement of energy expenditure was not due to any increase in physical activity (FIG. 29C). OX-treatment considerably decreased the amount of visceral fat (FIG. 29D). Further, body composition analysis at the end of the study, shown in Table 4, below, demonstrated that whole body fat mass was reduced by 55% in the OX-injected group. Vehicle-injected animals displayed 36% body fat, which was reduced to 27% in the experimental group. Lean mass or fluid content was not significantly different between the groups.

To evaluate changes associated with weight loss, OX-treated and untreated mice were compared at autopsy. Untreated mice fed a high fat diet had developed fatty livers, which were visibly paler in color than those from OX-treated mice (FIG. 29E). Oil Red O (ORO) staining of liver sections revealed the accumulation of triglycerides in the untreated group. Triglyceride measurements indicated that control mice on a high fat diet showed 152±18 μmol/g triglycerides within the liver compared to 91±7 μmol/g in OX-treated mice. Therefore, OX therapy had a desirable effect in reducing hepatic steatosis (FIG. 29E). iBAT from OX-treated mice appeared strikingly brown, indicating increased mitochondrial content (FIG. 29F). Microscopic examination of iBAT sections stained with mitoTracker indicated elevated mitochondrial content/activity in the OX-treated group relative to controls (FIG. 29G).

TABLE 4

| | | | | Lean mass | |
|---|---|---|---|---|---|
| | | | | fat mass | |
| | Body weight | Fat mass | Lean mass | ratio | % body fat |
| vehicle | 47.6 ± 1.1 | 17.5 ± 0.5 | 22.2 ± 1.7 | 1.2 ± 0.1 | 36 ± 1.8 |
| OX | 29.2 ± 0.8 | 7.9 ± 0.6 | 20.4 ± 1.3 | 2.58 ± 0.2 | 27 ± 1.4 |

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for inducing weight loss in a subject comprising administering to the subject a therapeutically effective amount of a pharmaceutical formulation comprising orexin or a biologically active fragment thereof and a pharmaceutically-acceptable carrier.

2. The method of claim 1, herein the subject is administered oxexin.

3. The method of claim 1, wherein the orexin or biologically active fragment is administered at a dose of about 1 mg/kg to about 100 mg/kg.

4. The method of claim 1, wherein the pharmaceutical formulation is administered to the subject 1-4 times per day.

5. The method of claim 1, wherein the pharmaceutical formulation is administered to the subject for at least one month.

6. The method of claim 1, wherein the subject is a human.

7. A method for treating diabetes comprising administering, to a subject diagnosed as having diabetes, a therapeutically effective amount of a pharmaceutical formulation comprising orexin or a biologically active fragment thereof and a pharmaceutically-acceptable carrier.

8. The method of claim 7, wherein the subject is administered oxexin.

9. The method of claim 7, wherein the orexin or biologically active fragment is administered at a dose of about 1 mg/kg to about 100 mg/kg.

10. The method of claim 7, wherein the pharmaceutical formulation is administered to the subject 1-4 times per day.

11. The method of claim 7, wherein the pharmaceutical formulation is administered to the subject for at least one month.

12. The method of claim 7, wherein the subject is a human.

13. A method of preventing weight gain in a subject, said method comprising administering to the subject a therapeutically effective amount of a pharmaceutical formulation comprising orexin or a biologically active fragment thereof and a pharmaceutically-acceptable carrier.

14. The method of claim 13, wherein the subject is administered oxexin.

15. The method of claim 13, wherein the orexin or biologically active fragment is administered at a dose of about 1 mg/kg to about 100 mg/kg.

16. The method of claim 13, wherein the pharmaceutical formulation is administered to the subject 1-4 times per day.

17. The method of claim 13, wherein the pharmaceutical formulation is administered to the subject for at least one month.

18. The method of claim 13, wherein the subject is a human.
